(19) United States
(12) Patent Application Publication
(10) Pub. No.: US 2011/0067143 A2
(43) Pub. Date: Mar. 17, 2011

(54) Nucleic Acid Molecules and Other Molecules Associated with Transcription in Plants and Uses Thereof for Plant Improvement

(76) Inventors: Thomas LA ROSA, Fenton, MO (US); Linda LUTFIYYA, St. Louis, MO (US); Yihua ZHOU, Ballwin, MO (US); David KOVALIC, Clayton, MO (US); Andrey BOUKHAROV, Melrose, MA (US); Ping LI, St. Peters, MO (US); Wei WU, Chesterfield, MO (US); Timothy CONNER, Loomis, CA (US); Jingdong LIU, Chesterfield, MO (US)

(21) Appl. No.: 10/438,246

(22) Filed: May 14, 2003

Related U.S. Application Data
(63) Continuation-in-part of application No. 09/565,306, filed on May 4, 2000, now abandoned.
Application No. 10/438,246 is a continuation-in-part of application No. 10/155,881, filed on May 22, 2002, now abandoned.
Application No. 10/438,246 is a continuation-in-part of application No. 09/816,660, filed on Mar. 26, 2001, now abandoned.
Application No. 09/816,660 is a continuation-in-part of application No. 09/733,089, filed on Dec. 11, 2000.
Application No. 10/438,246 is a continuation-in-part of application No. 10/424,599, filed on Apr. 28, 2003, now abandoned.
Application No. 10/424,599 is a continuation-in-part of application No. 09/985,678, filed on Nov. 5, 2001, now abandoned.
Application No. 09/985,678 is a continuation of application No. 09/304,517, filed on May 6, 1999, now abandoned.
(60) Provisional application No. 60/132,860, filed on May 7, 1999.

(51) Int. Cl. A01H 1/00; C12Q 1/68; C07H 21/04; C12N 9/22

(52) U.S. Cl. 800/278; 435/6; 435/199; 536/23.2

(57) ABSTRACT

Polynucleotides useful for improvement of plants are provided. In particular, polynucleotide sequences are provided from plant sources. Polypeptides encoded by the polynucleotide sequences are also provided. The disclosed polynucleotides and polypeptides find use in production of transgenic plants to produce plants having improved properties.

This application claims the benefit of U.S. application Ser. Nos. 09/938,294 filed Aug. 24, 2001, 10/155,881 filed May 22, 2002, 09/922,293 filed Aug. 6, 2001, 09/816,660 filed Mar. 26, 2001, 10/361,942 filed Feb. 10, 2003, and 09/828,073 filed Apr. 5, 2001, hereby incorporated by reference herein in their entirety.

### INCORPORATION OF SEQUENCE LISTING

Two copies of the sequence listing (Seq. Listing Copy 1 and Seq. Listing Copy 2) and a computer-readable form of the sequence listing, all on CD-ROMs, each containing the file named pa_{—}00563.rpt, which is 104,542,360 bytes (measured in MS-DOS) and was created on May 13, 2003, are herein incorporated by reference.

### INCORPORATION OF TABLE

Two copies of Table 1 (Table 1 Copy 1 and Table 1 Copy 2) all on CD-ROMs, each containing the file named pa_{—}00563.txt, which is 1,588,912 bytes (measured in MS-DOS) and was created on May 13, 2003, are herein incorporated by reference.

### FIELD OF THE INVENTION

Disclosed herein are inventions in the field of plant biochemistry and genetics. More specifically, this invention pertains to transcription factors, nucleic acid fragments encoding transcription factors, as well as plants and other organisms expressing transcription factors. This invention also relates to methods of using such agents, for example, in plant breeding.

### BACKGROUND OF THE INVENTION

Transcription is the essential first step in the conversion of the genetic information in the DNA into protein and the major point at which gene expression is controlled. Transcription of protein-coding genes is accomplished by the multisubunit enzyme RNA polymerase II and an ensemble of ancillary proteins called transcription factors. Basal (or general) transcription factors (a universal set of cellular proteins required for the transcription of all protein-coding genes) assist RNA polymerase II in aligning itself to the core region encompassing the transcription initiation site of genes and accurately initiating transcription. RNA polymerase II, basal transcription factors and an array of other proteins known as transcription co-factors comprise the basal transcription machinery that determines the constitutive level of gene transcription. Other transcription factors, termed gene-specific transcription factors, modulate transcription of a subset of protein-coding genes in response to specific environmental signals through binding to characteristic, cis-acting DNA sequence elements (motifs) and interactions with the basal transcription machinery. Cis-acting DNA sequence elements are often parts of larger regulatory entities called promoters or enhancers that confer a specific expression pattern to linked transcription units, their target genes. Collectively, these regions might bind several different gene-specific transcription factors each of which might contribute positively (activators) or negatively (repressors) to transcription initiation and rate. Protein-protein interactions between DNA-bound gene-specific transcription factors often result in synergistic or inhibitory regulatory effects. It is the sum of these combinatorial interactions that defines the transcriptional identity of a gene, turning genes on and off as appropriate for a specific biological context. In this manner, genes can be regulated, for example, tissue specifically, with a certain temporal or developmental pattern or become responsive to exogenous cues.

The identification of transcription factors and the subsequent modification of their activity may result in dramatic changes to a plant leading to plants with highly desirable, commercial traits. Root growth, tolerance to salt or cold stress, and flower characteristics are only some examples of plant traits that may be altered by modifying transcription factors.

Transcription factors may be identified by the presence of conserved functional domains. Typically, they are comprised of two domains that represent discrete functional entities. One of these is responsible for sequence-specific DNA recognition and binding (DNA binding domain); and the other facilitates communication with the basal transcription machinery, resulting in either the activation or repression of transcription initiation (transeffector domain). In addition, transcription factors also may contain oligomerization domains. This domain type may be adjacent to or overlap DNA binding domains and may act with them to effect the transcription factor's affinity for certain cis elements or other aspects of transcription factor activity. Nuclear localization signals that are characterized by a core peptide enriched in arginine and lysine may be present as well.

Such functional domains may be identified by examining the primary amino acid sequence of a putative transcription factor. For example, one class of transcription factors, the leucine zipper proteins, derive their name from the repeats they share of four or five leucine residues precisely seven amino acids apart. These domains provide hydrophobic faces through which leucine zipper proteins interact to form dimers. Zinc finger proteins are transcription factors so called because of the presence of repeated motifs of cysteine and histidine that are reported to fold up into a three-dimensional structure coordinated by a zinc ion.

Protein domains indicative of transcription factors have been described using Profile Hidden Markov Models (e.g. Profile HMM). Profile HMMs are based on position specific sequence information from multiple alignments. Different residues in a functional sequence are subject to different selective pressures. Multiple alignments of a sequence family reveal this in their pattern of conservation. Some positions are more conserved than others, and some regions of a multiple alignment are reported to tolerate insertions and deletions more than other regions.

An HMM (Hidden Markov Model) is used to statistically describe a protein family's consensus sequence. This statistical description can be used for sensitive and selective database searching. The model consists of a linear sequence of nodes with a “begin” state and an “end” state. A typical model can contain hundreds of nodes. Each node between the beginning and end state corresponds to a column in a multiple alignment. Each node in an HMM has a match state, an insert state, and a delete state with position-specific probabilities for transitioning into each of these states from the previous state. In addition to a transition probability, the match state also has position specific probabilities for emitting a particular residue. Likewise, the insert state has probabilities for inserting a residue at the position given by the node. There is also a chance that no residue is associated with a node. That probability is indicated by the probability of transitioning to the delete state. Both transition and emission probabilities can be generated from a multiple alignment of a family of sequences. An HMM can be aligned with a new sequence to determine the probability that the sequence belongs to the modeled family. The most probable path through the HMM (i.e. which transitions were taken and which residues were emitted at match and insert sites) taken to generate a sequence similar to the new sequence determines the similarity score.

Several available software packages implement profile HMMs or HMM-like models. These include SAM, HMMER, and HMMpro. Additionally, two collections of profile HMMs are currently available: the Pfam database and the PROSITE Profiles database.

Sequence similarity searches against known transcription factors or transcription factor domains resulting in statistically significant similarity between a putative and known transcription factor also provide strong evidence that both code for proteins with similar three dimensional structure and are thus likely to exhibit equivalent biochemical functions. The use of amino acid comparison methods-in particular those such as BLAST and FASTA which are sufficiently fast to search protein sequence databases (such as NCBI's non-redundant amino acid databases or Transfac which contains transcription factor domains have been used for such purposes). More rigorous algorithms such as that of the Frame+ program are also used.

Nucleic acid sequences and/or translations of nucleic acid sequences disclosed herein are cDNA and genomic sequences that have been queried for the presence of transcription factor functional domains. These sequences may be used in DNA constructs useful for imparting unique genetic properties into transgenic organisms. They may also be used to identify other transcription factor sequences.

### SUMMARY OF THE INVENTION

This invention provides a substantially purified nucleic acid molecule comprising nucleic acid sequences and the polypeptides encoded by such molecules from corn, soy, and rice. Nucleic acid sequences for the substantially purified nucleic acid molecules of the present invention are provided in the attached Sequence Listing as SEQ ID NO: 1-5429, SEQ ID NO: 10859-15800, SEQ ID NO: 20743-23549, and SEQ ID NO: 26357-29936. Amino acid sequences for the substantially purified polypeptides or fragment thereof of the present invention are provided as SEQ ID NO: 5430-10858, SEQ ID NO: 15801-20742, SEQ ID NO: 23550-26356, and SEQ ID NO: 29937-33516. Preferred subsets of the polynucleotides and polypeptides of this invention are useful for improvement of one or more important properties in plants.

The present invention also provides a method of producing a plant containing an overexpressed plant transcription factor comprising transforming said plant with a functional first nucleic acid molecule, wherein said first nucleic acid molecule comprises a promoter region, wherein said promoter region is linked to a structural region, wherein said structural region comprises a second nucleic acid molecule having a nucleic acid sequence selected from the group consisting of SEQ ID NO: 1-5429, SEQ ID NO: 10859-15800, SEQ ID NO: 20743-23549, and SEQ ID NO: 26357-29936; wherein said structural region is linked to a 3′ non-translated sequence that functions in the plant to cause termination of transcription of transcription and addition of polyadenylated ribonucleotides to a 3′ end of a mRNA molecule; and wherein said function first nucleic acid molecule results in overexpression of the plant transcription factor and then growing said plant.

The present invention also provides a method for determining a level or pattern of a plant transcription factor in a plant cell or plant tissue comprising incubating, under conditions permitting nucleic acid hybridization, a marker nucleic acid molecule, the marker nucleic acid molecule selected from the group of marker nucleic acid molecules which specifically hybridize to a nucleic acid molecule having the nucleic acid sequence selected from the group consisting of SEQ ID NO: 1-5429, SEQ ID NO: 10859-15800, SEQ ID NO: 20743-23549, and SEQ ID NO: 26357-29936 or complements thereof or fragments of either, with a complementary nucleic acid molecule obtained from the plant cell or plant tissue, wherein nucleic acid hybridization between the marker nucleic acid molecule and the complementary nucleic acid molecule obtained from the plant cell or plant tissue permits the detection of an mRNA for the enzyme; permitting hybridization between the marker nucleic acid molecule and the complementary nucleic acid molecule obtained from the plant cell or plant tissue; and then detecting the level or pattern of the complementary nucleic acid, wherein the detection of the complementary nucleic acid is predictive of the level or pattern of the plant transcription factor.

This invention also provides a transformed organism, particularly a transformed plant, preferably a transformed crop plant, comprising a recombinant DNA construct of the present invention.

### DETAILED DESCRIPTION OF THE INVENTION

The present invention provides polynucleotides, or nucleic acid molecules, representing DNA sequences and the polypeptides encoded by such polynucleotides from corn, soy, and rice. The polynucleotides and polypeptides of the present invention find a number of uses, for example in recombinant DNA constructs, in physical arrays of molecules, and for use as plant breeding markers. In addition, the nucleotide and amino acid sequences of the polynucleotides and polypeptides find use in computer based storage and analysis systems.

Depending on the intended use, the polynucleotides of the present invention may be present in the form of DNA, such as cDNA or genomic DNA, or as RNA, for example mRNA. The polynucleotides of the present invention may be single or double stranded and may represent the coding, or sense strand of a gene, or the non-coding, antisense, strand.

The polynucleotides of the present invention find particular use in generation of transgenic plants to provide for increased or decreased expression of the polypeptides encoded by the cDNA polynucleotides provided herein. As a result of such biotechnological applications, plants, particularly crop plants, having improved properties are obtained. Crop plants of interest in the present invention include, but are not limited to soy, cotton, canola, maize, wheat, sunflower, sorghum, alfalfa, barley, millet, rice, tobacco, fruit and vegetable crops, and turf grass. Of particular interest are uses of the disclosed polynucleotides to provide plants having improved yield resulting from improved utilization of key biochemical compounds, such as nitrogen, phosphorous and carbohydrate, or resulting from improved responses to environmental stresses, such as cold, heat, drought, salt, and attack by pests or pathogens. Polynucleotides of the present invention may also be used to provide plants having improved growth and development, and ultimately increased yield, as the result of modified expression of plant growth regulators or modification of cell cycle or photosynthesis pathways. Other traits of interest that may be modified in plants using polynucleotides of the present invention include flavonoid content, seed oil and protein quantity and quality, herbicide tolerance, and rate of homologous recombination.

The term “isolated” is used herein in reference to purified polynucleotide or polypeptide molecules. As used herein, “purified” refers to a polynucleotide or polypeptide molecule separated from substantially all other molecules normally associated with it in its native state. More preferably, a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “isolated” is also used herein in reference to polynucleotide molecules that are separated from nucleic acids which normally flank the polynucleotide in nature. Thus, polynucleotides fused to regulatory or coding sequences with which they are not normally associated, for example as the result of recombinant techniques, are considered isolated herein. Such molecules are considered isolated even when present, for example in the chromosome of a host cell, or in a nucleic acid solution. The terms “isolated” and “purified” as used herein are not intended to encompass molecules present in their native state.

As used herein a “transgenic” organism is one whose genome has been altered by the incorporation of foreign genetic material or additional copies of native genetic material, e.g. by transformation or recombination.

It is understood that the molecules of the invention may be labeled with reagents that facilitate detection of the molecule. As used herein, a label can be any reagent that facilitates detection, including fluorescent labels, chemical labels, or modified bases, including nucleotides with radioactive elements, e.g. ³²P, ³³P, ³⁵S or ¹²⁵I such as ³²P deoxycytidine-5′-triphosphate (³²PdCTP).

Polynucleotides of the present invention are capable of specifically hybridizing to other polynucleotides under certain circumstances. As used herein, two polynucleotides are said to be capable of specifically hybridizing to one another if the two molecules are capable of forming an anti-parallel, double-stranded nucleic acid structure. A nucleic acid molecule is said to be the “complement” of another nucleic acid molecule if the molecules exhibit complete complementarity. As used herein, molecules are said to exhibit “complete complementarity” when every nucleotide in each of the molecules is complementary to the corresponding nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are known to those skilled in the art and can be found, for example in Molecular Cloning: A Laboratory Manual, 3^{rd} edition Volumes 1, 2, and 3. J. F. Sambrook, D. W. Russell, and N. Irwin, Cold Spring Harbor Laboratory Press, 2000.

Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed. Appropriate stringency conditions which promote DNA hybridization are, for example, 6.0× sodium chloride/sodium citrate (SSC) at about 45° C., followed by a wash of 2.0×SSC at 50° C. Such conditions are known to those skilled in the art and can be found, for example in Current Protocols in Molecular Biology, John Wiley & Sons, N.Y. (1989). Salt concentration and temperature in the wash step can be adjusted to alter hybridization stringency. For example, conditions may vary from low stringency of about 2.0×SSC at 40° C. to moderately stringent conditions of about 2.0×SSC at 50° C. to high stringency conditions of about 0.2×SSC at 50° C.

As used herein “sequence identity” refers to the extent to which two optimally aligned polynucleotide or peptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as those in the BLAST suite of sequence analysis programs.

Polynucleotides

This invention provides polynucleotides comprising regions that encode polypeptides. The encoded polypeptides may be the complete protein encoded by the gene represented by the polynucleotide, or may be fragments of the encoded protein. Preferably, polynucleotides provided herein encode polypeptides constituting a substantial portion of the complete protein, and more preferentially, constituting a sufficient portion of the complete protein to provide the relevant biological activity.

A particularly preferred embodiment of the nucleic acid molecules of the present invention are plant nucleic acid molecules that comprise a nucleic acid sequence which encodes a transcription factor from one of the categories of transcription factors in Table 2 or fragment thereof, more preferably a nucleic acid molecule comprising a nucleic acid selected from the group consisting of SEQ ID NO: 1-5429, SEQ ID NO: 10859-15800, SEQ ID NO: 20743-23549, and SEQ ID NO: 26357-29936 or a nucleic acid molecule comprising a nucleic acid sequence which encodes a transcription factor from one of the categories of transcription factors in Table 2 or fragment thereof comprising an amino acid selected from the group consisting of SEQ ID NO: 1-5429, SEQ ID NO: 10859-15800, SEQ ID NO: 20743-23549, and SEQ ID NO: 26357-29936.

Polynucleotides of the present invention are generally used to impart such biological properties by providing for enhanced protein activity in a transgenic organism, preferably a transgenic plant, although in some cases, improved properties are obtained by providing for reduced protein activity in a transgenic plant. Reduced protein activity and enhanced protein activity are measured by reference to a wild type cell or organism and can be determined by direct or indirect measurement. Direct measurement of protein activity might include an analytical assay for the protein, per se, or enzymatic product of protein activity. Indirect assay might include measurement of a property affected by the protein. Enhanced protein activity can be achieved in a number of ways, for example by overproduction of mRNA encoding the protein or by gene shuffling. One skilled in the are will know methods to achieve overproduction of mRNA, for example by providing increased copies of the native gene or by introducing a construct having a heterologous promoter linked to the gene into a target cell or organism. Reduced protein activity can be achieved by a variety of mechanisms including antisense, mutation or knockout. Antisense RNA will reduce the level of expressed protein resulting in reduced protein activity as compared to wild type activity levels. A mutation in the gene encoding a protein may reduce the level of expressed protein and/or interfere with the function of expressed protein to cause reduced protein activity.

The polynucleotides of this invention represent cDNA sequences from corn, soy, and rice. Nucleic acid sequences of the polynucleotides of the present invention are provided herein as SEQ ID NO: 1-5429, SEQ ID NO: 10859-15800, SEQ ID NO: 20743-23549, and SEQ ID NO: 26357-29936.

A subset of the nucleic molecules of this invention includes fragments of the disclosed polynucleotides consisting of oligonucleotides of at least 15, preferably at least 16 or 17, more preferably at least 18 or 19, and even more preferably at least 20 or more, consecutive nucleotides. Such oligonucleotides are fragments of the larger molecules having a sequence selected from the group of polynucleotide sequences consisting of SEQ ID NO: 1-5429, SEQ ID NO: 10859-15800, SEQ ID NO: 20743-23549, and SEQ ID NO: 26357-29936, and find use, for example as probes and primers for detection of the polynucleotides of the present invention.

Also of interest in the present invention are variants of the polynucleotides provided herein. Such variants may be naturally occurring, including homologous polynucleotides from the same or a different species, or may be non-natural variants, for example polynucleotides synthesized using chemical synthesis methods, or generated using recombinant DNA techniques. With respect to nucleotide sequences, degeneracy of the genetic code provides the possibility to substitute at least one base of the protein encoding sequence of a gene with a different base without causing the amino acid sequence of the polypeptide produced from the gene to be changed. Hence, the DNA of the present invention may also have any base sequence that has been changed from SEQ ID NO: 1-5429, SEQ ID NO: 10859-15800, SEQ ID NO: 20743-23549, and SEQ ID NO: 26357-29936 by substitution in accordance with degeneracy of the genetic code.

Polynucleotides of the present invention that are variants of the polynucleotides provided herein will generally demonstrate significant identity with the polynucleotides provided herein. Of particular interest are polynucleotide homologs having at least about 60% sequence identity, at least about 70% sequence identity, at least about 80% sequence identity, at least about 85% sequence identity, and more preferably at least about 90%, 95% or even greater, such as 98% or 99% sequence identity with polynucleotide sequences described herein.

Nucleic acid molecules of the present invention also include homologues. Particularly preferred homologues are selected from the group consisting of Arabidopsis, alfalfa, barley, Brassica, broccoli, cabbage, citrus, cotton, garlic, oat, oilseed rape, onion, canola, flax, an ornamental plant, peanut, pepper, potato, rye, sorghum, strawberry, sugarcane, sugarbeet, tomato, wheat, poplar, pine, fir, eucalyptus, apple, lettuce, lentils, grape, banana, tea, turf grasses, sunflower, and Phaseolus.

In a preferred embodiment, nucleic acid molecules having SEQ ID NO: 1-5429, SEQ ID NO: 10859-15800, SEQ ID NO: 20743-23549, AND SEQ ID NO: 26357-29936 or complements thereof and fragments of either can be utilized to obtain such homologues.

Protein and Polypeptide Molecules

This invention also provides polypeptides encoded by polynucleotides of the present invention. Amino acid sequences of the polypeptides of the present invention are provided herein as SEQ ID NO: 5430-10858, SEQ ID NO: 15801-20742, SEQ ID NO: 23550-26356, and SEQ ID NO: 29937-33516.

As used herein, the term “protein molecule” or “peptide molecule” includes any molecule that comprises five or more amino acids. It is well known in the art that proteins may undergo modification, including post-translational modifications, such as, but not limited to, disulfide bond formation, glycosylation, phosphorylation, or oligomerization. Thus, as used herein, the term “protein molecule” or “peptide molecule” includes any protein molecule that is modified by any biological or non-biological process. The terms “amino acid” and “amino acids” refer to all naturally occurring L-amino acids. This definition is meant to include norleucine, norvaline, ornithine, homocysteine, and homoserine.

One or more of the protein or fragment of peptide molecules may be produced via chemical synthesis, or more preferably, by expressing in a suitable bacterial or eukaryotic host. Suitable methods for expression are well known to those skilled in the art.

A “protein fragment” is a peptide or polypeptide molecule whose amino acid sequence comprises a subset of the amino acid sequence of that protein. A protein or fragment thereof that comprises one or more additional peptide regions not derived from that protein is a “fusion” protein. Such molecules may be derivatized to contain carbohydrate or other moieties (such as keyhole limpet hemocyanin, etc.). Fusion protein or peptide molecules of the invention are preferably produced via recombinant means.

Another class of agents comprise protein or peptide molecules or fragments or fusions thereof comprising SEQ ID NO: 5430-10858, SEQ ID NO: 15801-20742, SEQ ID NO: 23550-26356, and SEQ ID NO: 29937-33516 in which conservative, non-essential or non-relevant amino acid residues have been added, replaced or deleted. Computerized means for designing modifications in protein structure are known in the art.

In a preferred embodiment, nucleic acid molecules having SEQ ID NO: 1-5429, SEQ ID NO: 10859-15800, SEQ ID NO: 20743-23549, and SEQ ID NO: 26357-29936 or polypeptide molecules having SEQ ID NO: 5430-10858, SEQ ID NO: 15801-20742, SEQ ID NO: 23550-26356, and SEQ ID NO: 29937-33516 or complements and fragments of any can be utilized to obtain such homologues.

Agents of the invention include proteins comprising at least about a contiguous 10 amino acid region more preferably comprising at least a contiguous 25, 40, 50, 75 or 125 amino acid region of a protein or fragment thereof of the present invention. In another preferred embodiment, the proteins of the present invention include a between about 10 and about 25 contiguous amino acid region, more preferably between about 20 and about 50 contiguous amino acid region and even more preferably between about 40 and about 80 contiguous amino acid region.

In a preferred embodiment the protein is selected from the group consisting of a plant, more preferably a maize, soybean, or rice transcription factor from the group consisting of Table 2. In another preferred embodiment, the protein comprises an amino acid sequence selected from the group consisting of SEQ ID NO: 5430-10858, SEQ ID NO: 15801-20742, SEQ ID NO: 23550-26356, and SEQ ID NO: 29937-33516.

Protein molecules of the present invention include homologues of proteins or fragments thereof comprising a protein sequence selected from SEQ ID NO: 5430-10858, SEQ ID NO: 15801-20742, SEQ ID NO: 23550-26356, and SEQ ID NO: 29937-33516 or fragment thereof or encoded by SEQ ID NO: 1-5429, SEQ ID NO: 10859-15800, SEQ ID NO: 20743-23549, and SEQ ID NO: 26357-29936 or fragments thereof. Preferred protein molecules of the invention include homologues of proteins or fragments having an amino acid sequence selected from the group consisting of SEQ ID NO: 5430-10858, SEQ ID NO: 15801-20742, SEQ ID NO: 23550-26356, and SEQ ID NO: 29937-33516 or fragment thereof.

A homologue protein may be derived from, but not limited to, alfalfa, barley, Brassica, broccoli, cabbage, citrus, cotton, garlic, oat, oilseed rape, onion, canola, flax, an ornamental plant, pea, peanut, pepper, potato, rye, sorghum, strawberry, sugarcane, sugar beet, tomato, wheat, poplar, pine, fir, eucalyptus, apple, lettuce, lentils, grape, banana, tea, turf grasses, sunflower, oil palm, Phaseolus etc. Particularly preferred species for use in the isolation of homologs would include, barley, cotton, oat, oilseed rape, canola, ornamentals, sugarcane, sugar beet, tomato, potato, wheat and turf grasses. Such a homologue can be obtained by any of a variety of methods. Most preferably, as indicated above, one or more of the disclosed sequences (such as SEQ ID NO: 1-5429, SEQ ID NO: 10859-15800, SEQ ID NO: 20743-23549, and SEQ ID NO: 26357-29936 or complements thereof) will be used in defining a pair of primers to isolate the homologue-encoding nucleic acid molecules from any desired species. Such molecules can be expressed to yield protein homologues by recombinant means.

Recombinant DNA Constructs

The present invention also encompasses the use of polynucleotides of the present invention in recombinant constructs, i.e. constructs comprising polynucleotides that are constructed or modified outside of cells and that join nucleic acids that are not found joined in nature. Using methods known to those of ordinary skill in the art, polypeptide encoding sequences of this invention can be inserted into recombinant DNA constructs that can be introduced into a host cell of choice for expression of the encoded protein, or to provide for reduction of expression of the encoded protein, for example by antisense or cosuppression methods. Potential host cells include both prokaryotic and eukaryotic cells. Of particular interest in the present invention is the use of the polynucleotides of the present invention for preparation of constructs for use in plant transformation.

In plant transformation, exogenous genetic material is transferred into a plant cell. By “exogenous” it is meant that a nucleic acid molecule, for example a recombinant DNA construct comprising a polynucleotide of the present invention, is produced outside the organism, e.g. plant, into which it is introduced. An exogenous nucleic acid molecule can have a naturally occurring or non-naturally occurring nucleotide sequence. One skilled in the art recognizes that an exogenous nucleic acid molecule can be derived from the same species into which it is introduced or from a different species. Such exogenous genetic material may be transferred into either monocot or dicot plants including, but not limited to, soy, cotton, canola, maize, teosinte, wheat, rice and Arabidopsis plants. Transformed plant cells comprising such exogenous genetic material may be regenerated to produce whole transformed plants.

Exogenous genetic material may be transferred into a plant cell by the use of a DNA vector or construct designed for such a purpose. A construct can comprise a number of sequence elements, including promoters, encoding regions, and selectable markers. Vectors are available which have been designed to replicate in both E. coli and A. tumefaciens and have all of the features required for transferring large inserts of DNA into plant chromosomes. Design of such vectors is generally within the skill of the art.

A construct will generally include a plant promoter to direct transcription of the protein-encoding region or the antisense sequence of choice. Numerous promoters, which are active in plant cells, have been described in the literature. These include the nopaline synthase (NOS) promoter and octopine synthase (OCS) promoters carried on tumor-inducing plasmids of Agrobacterium tumefaciens or caulimovirus promoters such as the Cauliflower Mosaic Virus (CaMV) 19S or 35S promoter (U.S. Pat. No. 5,352,605), and the Figwort Mosaic Virus (FMV) 35S-promoter (U.S. Pat. No. 5,378,619). These promoters and numerous others have been used to create recombinant vectors for expression in plants. Any promoter known or found to cause transcription of DNA in plant cells can be used in the present invention. Other useful promoters are described, for example, in U.S. Pat. Nos. 5,378,619; 5,391,725; 5,428,147; 5,447,858; 5,608,144; 5,614,399; 5,633,441, and 5,633,435, all of which are incorporated herein by reference.

In addition, promoter enhancers, such as the CaMV 35S enhancer or a tissue specific enhancer, may be used to enhance gene transcription levels. Enhancers often are found 5′ to the start of transcription in a promoter that functions in eukaryotic cells, but can often be inserted in the forward or reverse orientation 5′ or 3′ to the coding sequence. In some instances, these 5′ enhancing elements are introns. Deemed to be particularly useful as enhancers are the 5′ introns of the rice actin 1 and rice actin 2 genes. Examples of other enhancers which could be used in accordance with the invention include elements from octopine synthase genes, the maize alcohol dehydrogenase gene intron 1, elements from the maize shrunken 1 gene, the sucrose synthase intron, the TMV omega element, and promoters from non-plant eukaryotes.

DNA constructs can also contain one or more 5′ non-translated leader sequences which serve to enhance polypeptide production from the resulting mRNA transcripts. Such sequences may be derived from the promoter selected to express the gene or can be specifically modified to increase translation of the mRNA. Such regions may also be obtained from viral RNAs, from suitable eukaryotic genes, or from a synthetic gene sequence. For a review of optimizing expression of transgenes, see Koziel et al. (1996) Plant Mol. Biol. 32:393-405).

Constructs and vectors may also include, with the coding region of interest, a nucleic acid sequence that acts, in whole or in part, to terminate transcription of that region. One type of 3′ untranslated sequence which may be used is a 3′ UTR from the nopaline synthase gene (nos 3′) of Agrobacterium tumefaciens. Other 3′ termination regions of interest include those from a gene encoding the small subunit of a ribulose-1,5-bisphosphate carboxylase-oxygenase (rbcS), and more specifically, from a rice rbcS gene (U.S. Pat. No. 6,426,446), the 3′ UTR for the T7 transcript of Agrobacterium tumefaciens, the 3′ end of the protease inhibitor I or II genes from potato or tomato, and the 3′ region isolated from Cauliflower Mosaic Virus. Alternatively, one also could use a gamma coixin, oleosin 3 or other 3′ UTRs from the genus Coix (PCT Publication WO 99/58659).

Constructs and vectors may also include a selectable marker. Selectable markers may be used to select for plants or plant cells that contain the exogenous genetic material. Useful selectable marker genes include those conferring resistance to antibiotics such as kanamycin (nptII), hygromycin B (aph IV) and gentamycin (aac3 and aacC4) or resistance to herbicides such as glufosinate (bar or pat) and glyphosate (EPSPS). Examples of such selectable markers are illustrated in U.S. Pat. Nos. 5,550,318; 5,633,435; 5,780,708 and 6,118,047, all of which are incorporated herein by reference.

Constructs and vectors may also include a screenable marker. Screenable markers may be used to monitor transformation. Exemplary screenable markers include genes expressing a colored or fluorescent protein such as a luciferase or green fluorescent protein (GFP), a β-glucuronidase or uidA gene (GUS) which encodes an enzyme for which various chromogenic substrates are known or an R-locus gene, which encodes a product that regulates the production of anthocyanin pigments (red color) in plant tissues. Other possible selectable and/or screenable marker genes will be apparent to those of skill in the art.

Constructs and vectors may also include a transit peptide for targeting of a gene target to a plant organelle, particularly to a chloroplast, leucoplast or other plastid organelle (U.S. Pat. No. 5,188,642).

For use in Agrobacterium mediated transformation methods, constructs of the present invention will also include T-DNA border regions flanking the DNA to be inserted into the plant genome to provide for transfer of the DNA into the plant host chromosome as discussed in more detail below. An exemplary plasmid that finds use in such transformation methods is pMON18365, a T-DNA vector that can be used to clone exogenous genes and transfer them into plants using Agrobacterium-mediated transformation. See US Patent Application 20030024014, herein incorporated by reference. This vector contains the left border and right border sequences necessary for Agrobacterium transformation. The plasmid also has origins of replication for maintaining the plasmid in both E. coli and Agrobacterium tumefaciens strains.

A candidate gene is prepared for insertion into the T-DNA vector, for example using well-known gene cloning techniques such as PCR. Restriction sites may be introduced onto each end of the gene to facilitate cloning. For example, candidate genes may be amplified by PCR techniques using a set of primers. Both the amplified DNA and the cloning vector are cut with the same restriction enzymes, for example, NotI and PstI. The resulting fragments are gel-purified, ligated together, and transformed into E. coli. Plasmid DNA containing the vector with inserted gene may be isolated from E. coli cells selected for spectinomycin resistance, and the presence of the desired insert verified by digestion with the appropriate restriction enzymes. Undigested plasmid may then be transformed into Agrobacterium tumefaciens using techniques well known to those in the art, and transformed Agrobacterium cells containing the vector of interest selected based on spectinomycin resistance. These and other similar constructs useful for plant transformation may be readily prepared by one skilled in the art.

Transformation Methods and Transpenic Plants

Methods and compositions for transforming bacteria and other microorganisms are known in the art. See for example Molecular Cloning: A Laboratory Manual, 3^{rd} edition Volumes 1, 2, and 3. J. F. Sambrook, D. W. Russell, and N. Irwin, Cold Spring Harbor Laboratory Press, 2000.

Technology for introduction of DNA into cells is well known to those of skill in the art. Methods and materials for transforming plants by introducing a transgenic DNA construct into a plant genome in the practice of this invention can include any of the well-known and demonstrated methods including electroporation as illustrated in U.S. Pat. No. 5,384,253, microprojectile bombardment as illustrated in U.S. Pat. Nos. 5,015,580; 5,550,318; 5,538,880; 6,160,208; 6,399,861 and 6,403,865, Agrobacterium-mediated transformation as illustrated in U.S. Pat. Nos. 5,635,055; 5,824,877; 5,591,616; 5,981,840 and 6,384,301, and protoplast transformation as illustrated in U.S. Pat. No. 5,508,184, all of which are incorporated herein by reference.

Any of the polynucleotides of the present invention may be introduced into a plant cell in a permanent or transient manner in combination with other genetic elements such as vectors, promoters enhancers etc. Further any of the polynucleotides of the present invention may be introduced into a plant cell in a manner that allows for production of the polypeptide or fragment thereof encoded by the polynucleotide in the plant cell, or in a manner that provides for decreased expression of an endogenous gene and concomitant decreased production of protein.

It is also to be understood that two different transgenic plants can also be mated to produce offspring that contain two independently segregating added, exogenous genes. Selfing of appropriate progeny can produce plants that are homozygous for both added, exogenous genes that encode a polypeptide of interest. Back-crossing to a parental plant and out-crossing with a non-transgenic plant are also contemplated, as is vegetative propagation.

Expression of the polynucleotides of the present invention and the concomitant production of polypeptides encoded by the polynucleotides is of interest for production of transgenic plants having improved properties, particularly, improved properties which result in crop plant yield improvement. Expression of polypeptides of the present invention in plant cells may be evaluated by specifically identifying the protein products of the introduced genes or evaluating the phenotypic changes brought about by their expression. It is noted that when the polypeptide being produced in a transgenic plant is native to the target plant species, quantitative analyses comparing the transformed plant to wild type plants may be required to demonstrate increased expression of the polypeptide of this invention.

Assays for the production and identification of specific proteins make use of various physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures may also be used to identify the expression of proteins by their functionality, particularly where the expressed protein is an enzyme capable of catalyzing chemical reactions involving specific substrates and products. These reactions may be measured, for example in plant extracts, by providing and quantifying the loss of substrates or the generation of products of the reactions by physical and/or chemical procedures.

In many cases, the expression of a gene product is determined by evaluating the phenotypic results of its expression. Such evaluations may be simply as visual observations, or may involve assays. Such assays may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks.

Plants with decreased expression of a gene of interest can also be achieved through the use of polynucleotides of the present invention, for example by expression of antisense nucleic acids, or by identification of plants transformed with sense expression constructs that exhibit cosuppression effects.

Antisense approaches are a way of preventing or reducing gene function by targeting the genetic material as disclosed in U.S. Pat. Nos. 4,801,540; 5,107,065; 5,759,829; 5,910,444; 6,184,439; and 6,198,026, all of which are incorporated herein by reference. The objective of the antisense approach is to use a sequence complementary to the target gene to block its expression and create a mutant cell line or organism in which the level of a single chosen protein is selectively reduced or abolished. Antisense techniques have several advantages over other ‘reverse genetic’ approaches. The site of inactivation and its developmental effect can be manipulated by the choice of promoter for antisense genes or by the timing of external application or microinjection. Antisense can manipulate its specificity by selecting either unique regions of the target gene or regions where it shares homology to other related genes.

The principle of regulation by antisense RNA is that RNA that is complementary to the target mRNA is introduced into cells, resulting in specific RNA:RNA duplexes being formed by base pairing between the antisense substrate and the target. Under one embodiment, the process involves the introduction and expression of an antisense gene sequence. Such a sequence is one in which part or all of the normal gene sequences are placed under a promoter in inverted orientation so that the ‘wrong’ or complementary strand is transcribed into a noncoding antisense RNA that hybridizes with the target mRNA and interferes with its expression. An antisense vector is constructed by standard procedures and introduced into cells by transformation, transfection, electroporation, microinjection, infection, etc. The type of transformation and choice of vector will determine whether expression is transient or stable. The promoter used for the antisense gene may influence the level, timing, tissue, specificity, or inducibility of the antisense inhibition.

As used herein “gene suppression” means any of the well-known methods for suppressing expression of protein from a gene including sense suppression, anti-sense suppression and RNAi suppression. In suppressing genes to provide plants with a desirable phenotype, anti-sense and RNAi gene suppression methods are preferred. More particularly, for a description of anti-sense regulation of gene expression in plant cells see U.S. Pat. No. 5,107,065 and for a description of RNAi gene suppression in plants by transcription of a dsRNA see U.S. Pat. No. 6,506,559, U.S. Patent Application Publication No. 2002/0168707 A1, and U.S. patent application Ser. No. 09/423,143 (see WO 98/53083), 09/127,735 (see WO 99/53050) and 09/084,942 (see WO 99/61631), all of which are incorporated herein by reference. Suppression of an gene by RNAi can be achieved using a recombinant DNA construct having a promoter operably linked to a DNA element comprising a sense and anti-sense element of a segment of genomic DNA of the gene, e.g., a segment of at least about 23 nucleotides, more preferably about 50 to 200 nucleotides where the sense and anti-sense DNA components can be directly linked or joined by an intron or artificial DNA segment that can form a loop when the transcribed RNA hybridizes to form a hairpin structure. For example, genomic DNA from a polymorphic locus of SEQ ID NO: 1-5429, SEQ ID NO: 10859-15800, SEQ ID NO: 20743-23549, AND SEQ ID NO: 26357-29936 can be used in a recombinant construct for suppression of a cognate gene by RNAi suppression.

Insertion mutations created by transposable elements may also prevent gene function. For example, in many dicot plants, transformation with the T-DNA of Agrobacterium may be readily achieved and large numbers of transformants can be rapidly obtained. Also, some species have lines with active transposable elements that can efficiently be used for the generation of large numbers of insertion mutations, while some other species lack such options. Mutant plants produced by Agrobacterium or transposon mutagenesis and having altered expression of a polypeptide of interest can be identified using the polynucleotides of the present invention. For example, a large population of mutated plants may be screened with polynucleotides encoding the polypeptide of interest to detect mutated plants having an insertion in the gene encoding the polypeptide of interest.

Polynucleotides of the present invention may be used in site-directed mutagenesis. Site-directed mutagenesis may be utilized to modify nucleic acid sequences, particularly as it is a technique that allows one or more of the amino acids encoded by a nucleic acid molecule to be altered (e.g., a threonine to be replaced by a methionine). Three basic methods for site-directed mutagenesis are often employed. These are cassette mutagenesis, primer extension, and methods based upon PCR.

In addition to the above-discussed procedures, practitioners are familiar with the standard resource materials which describe specific conditions and procedures for the construction, manipulation and isolation of macromolecules (e.g., DNA molecules, plasmids, etc.), generation of recombinant organisms and the screening and isolating of clones.

Arrays

The polynucleotide or polypeptide molecules of this invention may also be used to prepare arrays of target molecules arranged on a surface of a substrate. The target molecules are preferably known molecules, e.g. polynucleotides (including oligonucleotides) or polypeptides, which are capable of binding to specific probes, such as complementary nucleic acids or specific antibodies. The target molecules are preferably immobilized, e.g. by covalent or non-covalent bonding, to the surface in small amounts of substantially purified and isolated molecules in a grid pattern. By immobilized is meant that the target molecules maintain their position relative to the solid support under hybridization and washing conditions. Target molecules are deposited in small footprint, isolated quantities of “spotted elements” of preferably single-stranded polynucleotide preferably arranged in rectangular grids in a density of about 30 to 100 or more, e.g. up to about 1000, spotted elements per square centimeter. In addition in preferred embodiments arrays comprise at least about 100 or more, e.g. at least about 1000 to 5000, distinct target polynucleotides per unit substrate. Where detection of transcription for a large number of genes is desired, the economics of arrays favors a high density design criteria provided that the target molecules are sufficiently separated so that the intensity of the indicia of a binding event associated with highly expressed probe molecules does not overwhelm and mask the indicia of neighboring binding events. For high-density microarrays each spotted element may contain up to about 10⁷ or more copies of the target molecule, e.g. single stranded cDNA, on glass substrates or nylon substrates.

Arrays of this invention can be prepared with molecules from a single species, preferably a plant species, or with molecules from other species, particularly other plant species. Arrays with target molecules from a single species can be used with probe molecules from the same species or a different species due to the ability of cross species homologous genes to hybridize. It is generally preferred for high stringency hybridization that the target and probe molecules are from the same species.

In preferred aspects of this invention the organism of interest is a plant and the target molecules are polynucleotides or oligonucleotides with nucleic acid sequences having at least 80 percent sequence identity to a corresponding sequence of the same length in a polynucleotide having a sequence selected from the group consisting of SEQ ID NO: 1-5429, SEQ ID NO: 10859-15800, SEQ ID NO: 20743-23549, and SEQ ID NO: 26357-29936 or complements thereof. In other preferred aspects of the invention at least 10% of the target molecules on an array have at least 15, more preferably at least 20, consecutive nucleotides of sequence having at least 80%, more preferably up to 100%, identity with a corresponding sequence of the same length in a polynucleotide having a sequence selected from the group consisting of SEQ ID NO: 1-5429, SEQ ID NO: 10859-15800, SEQ ID NO: 20743-23549, and SEQ ID NO: 26357-29936 or complements or fragments thereof.

Such arrays are useful in a variety of applications, including gene discovery, genomic research, molecular breeding and bioactive compound screening. One important use of arrays is in the analysis of differential gene transcription, e.g. transcription profiling where the production of mRNA in different cells, normally a cell of interest and a control, is compared and discrepancies in gene expression are identified. In such assays, the presence of discrepancies indicates a difference in gene expression levels in the cells being compared. Such information is useful for the identification of the types of genes expressed in a particular cell or tissue type in a known environment. Such applications generally involve the following steps: (a) preparation of probe, e.g. attaching a label to a plurality of expressed molecules; (b) contact of probe with the array under conditions sufficient for probe to bind with corresponding target, e.g. by hybridization or specific binding; (c) removal of unbound probe from the array; and (d) detection of bound probe.

A probe may be prepared with RNA extracted from a given cell line or tissue. The probe may be produced by reverse transcription of mRNA or total RNA and labeled with radioactive or fluorescent labeling. A probe is typically a mixture containing many different sequences in various amounts, corresponding to the numbers of copies of the original mRNA species extracted from the sample.

The initial RNA sample for probe preparation will typically be derived from a physiological source. The physiological source may be selected from a variety of organisms, with physiological sources of interest including single celled organisms such as yeast and multicellular organisms, including plants and animals, particularly plants, where the physiological sources from multicellular organisms may be derived from particular organs or tissues of the multicellular organism, or from isolated cells derived from an organ, or tissue of the organism. The physiological sources may also be multicellular organisms at different developmental stages (e.g., 10-day-old seedlings), or organisms grown under different environmental conditions (e.g., drought-stressed plants) or treated with chemicals.

In preparing the RNA probe, the physiological source may be subjected to a number of different processing steps, where such processing steps might include tissue homogenation, cell isolation and cytoplasmic extraction, nucleic acid extraction and the like, where such processing steps are known to the those of skill in the art. Methods of isolating RNA from cells, tissues, organs or whole organisms are known to those of skill in the art.

Computer Based Systems and Methods

The sequence of the molecules of this invention can be provided in a variety of media to facilitate use thereof. Such media can also provide a subset thereof in a form that allows a skilled artisan to examine the sequences. In a preferred embodiment, 20, preferably 50, more preferably 100, even more preferably 200 or more of the polynucleotide and/or the polypeptide sequences of the present invention can be recorded on computer readable media. As used herein, “computer readable media” refers to any medium that can be read and accessed directly by a computer. Such media include, but are not limited to: magnetic storage media, such as floppy discs, hard disc, storage medium, and magnetic tape: optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising a computer readable medium having recorded thereon a nucleotide sequence of the present invention.

As used herein, “recorded” refers to a process for storing information on computer readable media. A skilled artisan can readily adopt any of the presently known methods for recording information on computer readable media to generate media comprising the nucleotide sequence information of the present invention. A variety of data storage structures are available to a skilled artisan for creating a computer readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable media. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of data processor structuring formats (e.g., text file or database) in order to obtain a computer readable medium having recorded thereon the nucleotide sequence information of the present invention.

By providing one or more of polynucleotide or polypeptide sequences of the present invention in a computer readable medium, a skilled artisan can routinely access the sequence information for a variety of purposes. The examples which follow demonstrate how software which implements the BLAST and BLAZE search algorithms on a Sybase system can be used to identify open reading frames (ORFs) within the genome that contain homology to ORFs or polypeptides from other organisms. Such ORFs are polypeptide encoding fragments within the sequences of the present invention and are useful in producing commercially important polypeptides such as enzymes used in amino acid biosynthesis, metabolism, transcription, translation, RNA processing, nucleic acid and a protein degradation, protein modification, and DNA replication, restriction, modification, recombination, and repair.

The present invention further provides systems, particularly computer-based systems, which contain the sequence information described herein. Such systems are designed to identify commercially important fragments of the nucleic acid molecule of the present invention. As used herein, “a computer-based system” refers to the hardware, software, and memory used to analyze the sequence information of the present invention. A skilled artisan can readily appreciate that any one of the currently available computer-based systems are suitable for use in the present invention.

As indicated above, the computer-based systems of the present invention comprise a database having stored therein a nucleotide sequence of the present invention and the necessary hardware and software for supporting and implementing a homology search. As used herein, “database” refers to memory system that can store searchable nucleotide sequence information. As used herein “query sequence” is a nucleic acid sequence, or an amino acid sequence, or a nucleic acid sequence corresponding to an amino acid sequence, or an amino acid sequence corresponding to a nucleic acid sequence, that is used to query a collection of nucleic acid or amino acid sequences. As used herein, “homology search” refers to one or more programs which are implemented on the computer-based system to compare a query sequence, i.e., gene or peptide or a conserved region (motif), with the sequence information stored within the database. Homology searches are used to identify segments and/or regions of the sequence of the present invention that match a particular query sequence. A variety of known searching algorithms are incorporated into commercially available software for conducting homology searches of databases and computer readable media comprising sequences of molecules of the present invention.

Commonly preferred sequence length of a query sequence is from about 10 to 100 or more amino acids or from about 20 to 300 or more nucleotide residues. There are a variety of motifs known in the art. Protein motifs include, but are not limited to, enzymatic active sites and signal sequences. An amino acid query is converted to all of the nucleic acid sequences that encode that amino acid sequence by a software program, such as TBLASTN, which is then used to search the database. Nucleic acid query sequences that are motifs include, but are not limited to, promoter sequences, cis elements, hairpin structures and inducible expression elements (protein binding sequences).

Thus, the present invention further provides an input device for receiving a query sequence, a memory for storing sequences (the query sequences of the present invention and sequences identified using a homology search as described above) and an output device for outputting the identified homologous sequences. A variety of structural formats for the input and output presentations can be used to input and output information in the computer-based systems of the present invention. A preferred format for an output presentation ranks fragments of the sequence of the present invention by varying degrees of homology to the query sequence. Such presentation provides a skilled artisan with a ranking of sequences that contain various amounts of the query sequence and identifies the degree of homology contained in the identified fragment.

Having now generally described the invention, the same will be more readily understood through reference to the following examples which are provided by way of illustration, and are not intended to be limiting of the present invention, unless specified.

### EXAMPLE 1

This example illustrates the construction of the rice genomic library. BACs are stable, non-chimeric cloning systems having genomic fragment inserts (100-300 kb) and their DNA can be prepared for most types of experiments including DNA sequencing. BAC vector, pBeloBAC11, is derived from the endogenous E. coli F-factor plasmid, which contains genes for strict copy number control and unidirectional origin of DNA replication. Additionally, pBeloBAC11 has three unique restriction enzyme sites (Hind III, Bam HI and Sph I) located within the LacZ gene that can be used as cloning sites for megabase-size plant DNA. Indigo, another BAC vector contains Hind III and Eco RI cloning sites. This vector also contains a random mutation in the LacZ gene that allows for darker blue colonies.

As an alternative, the P1-derived artificial chromosome (PAC) can be used as a large DNA fragment cloning vector (Ioannou et al., Nature Genet. 6:84-89 (1994; Suzuki et al., Gene 199:133-137 (1997). The PAC vector has most of the features of the BAC system, but also contains some of the elements of the bacteriophage P1 cloning system.

BAC libraries are generated by ligating size-selected restriction digested DNA with pBeloBAC11 followed by electroporation into E. coli. BAC library construction and characterization is extremely efficient when compared to YAC (yeast artificial chromosome) library construction and analysis, particularly because of the chimerism associated with YACs and difficulties associated with extracting YAC DNA.

There are general methods for preparing megabase-size DNA from plants. For example, the protoplast method yields megabase-size DNA of high quality with minimal breakage. The process involves preparing young leaves that are manually feathered with a razor-blade before being incubated for four to five hours with cell-wall-degrading enzymes. The second method developed by Zhange et al., Plant J 7:175-184 (1995), is a universal nuclei method that works well for several divergent plant taxa. Fresh or frozen tissue is homogenized with a blender or mortar and pestle. Nuclei are then isolated and embedded. DNA prepared by the nucleic method is often more concentrated and is reported to contain lower amounts of chloroplast DNA than the protoplast method.

Once protoplasts or nuclei are produced, they are embedded in an agarose matrix as plugs or microbeads. The agarose provides a support matrix to prevent shearing of the DNA while allowing enzymes and buffers to diffuse into the DNA. The DNA is purified and manipulated in the agarose and is stable for more than one year at 4° C.

Once high molecular weight DNA has been prepared, it is fragmented to the desired size range. In general, DNA fragmentation utilizes two general approaches, 1) physical shearing and 2) partial digestion with a restriction enzyme that cuts relatively frequently within the genome. Since physical shearing is not dependent upon the frequency and distribution of particular restriction enzymes sites, this method should yield the most random distribution of DNA fragments. However, the ends of the sheared DNA fragments must be repaired and cloned directly or restriction enzyme sites added by the addition of synthetic linkers. Because of the subsequent steps required to clone DNA fragmented by shearing, most protocols fragment DNA by partial restriction enzyme digestion. The advantage of partial restriction enzyme digestion is that no further enzymatic modification of the ends of the restriction fragments is necessary. Four common techniques that can be used to achieve reproducible partial digestion of megabase-size DNA are 1) varying the concentration of the restriction enzyme, 2) varying the time of incubation with the restriction enzyme 3) varying the concentration of an enzyme cofactor (e.g., Mg²⁺) and 4) varying the ratio of endonuclease to methylase.

There are three cloning sites in pBeloBAC11, but only Hind III and Bam HI produce 5′ overhangs for easy vector dephosphorylation. These two restriction enzymes are primarily used to construct BAC libraries. The optimal partial digestion conditions for megabase-size DNA are determined by wide and narrow window digestions. To optimize the optimum amount of Hind III, 1, 2, 3, 10, and 5-units of enzyme are each added to 50 ml aliquots of microbeads and incubated at 37° C. for 20 minutes.

After partial digestion of megabase-size DNA, the DNA is run on a pulsed-field gel, and DNA in a size range of 100-500 kb is excised from the gel. This DNA is ligated to the BAC vector or subjected to a second size selection on a pulsed field gel under different running conditions. Studies have previously reported that two rounds of size selection can eliminate small DNA fragments co-migrating with the selected range in the first pulse-field fractionation. Such a strategy results in an increase in insert sizes and a more uniform insert size distribution. A practical approach to performing size selections is to first test for the number of clones/microliter of ligation and insert size from the first size selected material. If the numbers are good (500 to 2000 white colony/microliter of ligation) and the size range is also good (50 to 300 kb) then a second size selection is practical. When performing a second size selection one expects an 80 to 95% decrease in the number of recombinant clones per transformation.

Twenty to two hundred nanograms of the size-selected DNA are ligated to dephosphorylated BAC vector (molar ratio of 10 to 1 in BAC vector excess). Most BAC libraries use a molar ratio of 5 to 15:1 (size selected DNA: BAC vector).

Transformation is carried out by electroporation and the transformation efficiency for BACs is about 40 to 1,500 transformants from one microliter of ligation product or 20 to 1000 transformants/ng DNA.

Several tests can be carried out to determine the quality of a BAC library. Three basic tests to evaluate the quality include: the genome coverage of a BAC library-average insert size, average number of clones hybridizing with single copy probes and chloroplast DNA content.

The determination of the average insert size of the library is assessed in two ways. First, during library construction every ligation is tested to determine the average insert size by assaying 20-50 BAC clones per ligation. DNA is isolated from recombinant clones using a standard mini preparation protocol, digested with Not I to free the insert from the BAC vector and then sized using pulsed field gel electrophoresis (Maule, Molecular Biotechnology 9:107-126 (1998)).

To determine the genome coverage of the library, it is screened with single copy RFLP markers distributed randomly across the genome by hybridization. Microtiter plates containing BAC clones are spotted onto Hybond membranes. Bacteria from 48 or 72 plates are spotted twice onto one membrane resulting in 18,000 to 27,648 unique clones on each membrane in either a 4×4 or 5×5 orientation. Since each clone is present twice, false positives are easily eliminated and true positives are easily recognized and identified.

Finally, the chloroplast DNA content in the BAC library is estimated by hybridizing three chloroplast genes spaced evenly across the chloroplast genome to the library on high density hybridization filters.

There are strategies for isolating rare sequences within the genome. For example, higher plant genomes can range in size from 100 Mb/1C (Arabidopsis) to 15,966 Mb/C (Triticum aestivum), (Arumuganathan and Earle, Plant Mol Bio Rep. 9: 208-219 (1991)). The number of clones required to achieve a given probability that any DNA sequence will be represented in a genomic library is N=ln(1−P))/(ln(1−L/G)) where N is the number of clones required, P is the probability desired to get the target sequence, L is the length of the average clone insert in base pairs and G is the haploid genome length in base pairs (Clarke et al., Cell 9:91-100 (1976)).

The rice BAC library of the present invention is constructed in the pBeloBAC11 or similar vector. Inserts are generated by partial Eco RI digestion or other enzymatic digestion of DNA.

### EXAMPLE 2

This example serves to illustrate how the genomic sequences are sequenced and combined into contigs. Basic methods can be used for DNA sequencing and are well known to one skilled in the art. Automation and advances in technology such as the replacement of radioisotopes with fluorescence-based sequencing have reduced the effort required to sequence DNA. Automated sequencers are available from, for example, Pharmacia Biotech, Inc., Piscataway, N.J. (Pharmacia ALF), LI-COR, Inc., Lincoln, Nebr. (LI-COR 4,000) and Millipore, Bedford, Mass. (Millipore BaseStation).

In addition, advances in capillary gel electrophoresis have also reduced the effort required to sequence DNA and such advances provide a rapid high resolution approach for sequencing DNA samples. The 3700 DNA Sequencer (Perkin-Elmer Corp., Applied Biosystems Div., Foster City, Calif.) is a machine that uses this technology.

A number of sequencing techniques are known in the art, including fluorescence-based sequencing methodologies. These methods have the detection, automation and instrumentation capability necessary for the analysis of large volumes of sequence data. With these types of automated systems, fluorescent dye-labeled sequence reaction products are detected and data entered directly into the computer, producing a chromatogram that is subsequently viewed, stored, and analyzed using the corresponding software programs. These methods are known to those of skill in the art and have been described and reviewed.

PHRED is used to call the bases from the sequence trace files. Phred uses Fourier methods to examine the four base traces in the region surrounding each point in the data set in order to predict a series of evenly spaced predicted locations. That is, it determines where the peaks would be centered if there were no compressions, dropouts, or other factors shifting the peaks from their “true” locations. Next, PHRED examines each trace to find the centers of the actual, or observed peaks and the areas of these peaks relative to their neighbors. The peaks are detected independently along each of the four traces so many peaks overlap. A dynamic programming algorithm is used to match the observed peaks detected in the second step with the predicted peak locations found in the first step.

After the base calling is completed, contaminating sequences (e.g., E. coli) are removed, and BAC vector and sub-cloning vectors sequence segments with >30 bases are trimmed and constraints are made for the assembler. Rice contigs are assembled using CAP3.

A two-step re-assembly process is employed to reduce sequence redundancies caused by overlaps between BAC clones. In the first step, BAC clones are grouped into clusters based on overlaps between contig sequences from different BACs. These overlaps are identified by comparing each sequence in the dataset against every other sequence, by BLASTN. BACs containing overlaps greater than 5,000 base pairs in length and greater than 94% in sequence identity are put into the same cluster. Repetitive sequences are masked prior to this procedure to avoid false joining by repetitive elements present in the genome. In the second step, sequences from each BAC cluster are assembled by PHRAP.longread, which is able to handle very long sequences. A minimum match is set at 100 bp and a minimum score is set at 600 as a threshold to join input contigs into longer contigs.

Oryza sativa contigs are assembled using PANGEA clustering tools and PHRAP. PANGEA clustering tools are a series of scripts that group sequences (clusters) by comparing pairs of sequences for overlapping bases. The overlap is determined using the following high stringency parameters: word size=8; window size=60; and identity is 93%. Each of the clusters is then assembled using PHRAP. This step results in islands. The next step is to combine the islands together to collapse the contig number even further. Default, less stringent parameters, are used in this step: minimum match=14, minimum score=30; and the penalty is −2.

### EXAMPLE 3

This example illustrates the identification of genes within rice genomic contig libraries as assembled above. The genes and partial genes embedded in such contigs are identified through a series of bioinformatic analyses. The tools to define genes fall into two categories: homology-based and predictive-based methods. Homology-based searches (e.g., GAP2, BLASTX supplemented by NAP and TBLASTX) detect conserved sequences during comparisons of DNA sequences or hypothetically translated protein sequences to public and/or proprietary DNA and protein databases. Existence of an Oryza sativa gene is inferred if significant sequence similarity extends over the majority of the target gene. Since homology-based methods may overlook genes unique to Oryza sativa, for which homologous nucleic acid molecules have not yet been identified in databases, gene prediction programs are also used. Predictive methods employed in the definition of the Oryza sativa genes include the use of the GenScan gene predictive software program. In general terms, GenScan infers the presence and extent of a gene through a search for “gene-like” grammar.

The homology-based methods used to define the Oryza sativa gene set include BLASTX supplemented by NAP. NAP is part of the Analysis and Annotation Tool (AAT) for Finding Genes in Genomic Sequences. The AAT package includes two sets of programs, one set DPS/NAP (referred to as “NAP”) for comparing the query sequence with a protein database, and the other set DDS/GAP2 (referred to as “GAP2”) for comparing the query sequence with a cDNA database. Each set contains a fast database search program and a rigorous alignment program. The database search program quickly identifies regions of the query sequence that are similar to a database sequence. Then the alignment program constructs an optimal alignment for each region and the database sequence. The alignment program also reports the coordinates of exons in the query sequence.

The NAP program computes a global alignment of a DNA sequence and a protein sequence without penalizing terminal gaps. NAP handles frameshifts and long introns in the DNA sequence. The program delivers the alignment in linear space; so long sequences can be aligned. It makes use of splice site consensuses in alignment computation. Both strands of the DNA sequence are compared with the protein sequence and one of the two alignments with the larger score is reported.

NAP takes a nucleotide sequence, translates it in three forward reading frames and three reverse complement reading frames, and then compares the six translations against a protein sequence database (e.g. the non-redundant protein (i.e., nr-aa) database maintained by the National Center for Biotechnology Information as part of GenBank and available at the web site: www.ncbi.nlm.nih.gov).

The second homology-based method used for gene discovery is BLASTX hits extended with the NAP software package. BLASTX is run with the Oryza sativa genomic contigs as queries against the GenBank non-redundant protein data library identified as “nr.aa”. NAP is used to better align the amino acid sequences as compared to the genomic sequence. NAP extends the match in regions where BLASTX has identified high-scoring-pairs (HSPs), predicts introns, and then links the exons into a single ORF prediction. Experience suggests that NAP tends to mispredict the first exon. The NAP parameters are:

gap extension penalty=1

gap open penalty=15

gap length for constant penalty=25

min exon length (in aa)=7

minimum total length of all exons in a gene (in nucleotide)=200

homology >40%

The NAP alignment score and GenBank reference number for best match are reported for each contig for which there is a NAP hit.

The GenScan program is “trained” with Arabidopsis thaliana characteristics. Though better than the “off-the-shelf” version, the GenScan trained to identify Oryza sativa and Arabidopsis thaliana genes proved more proficient at predicting exons than predicting full-length genes. Predicting full-length genes is compromised by point mutations in the unfinished contigs, as well as by the short length of the contigs relative to the typical length of a gene. Due to the errors found in the full-length gene predictions by GenScan, inclusion of GenScan-predicted genes is limited to those genes and exons whose probabilities are above a conservative probability threshold. The GenScan parameters are:

weighted mean GenScan P value>0.4

mean GenScan T value>0

mean GenScan Coding score>50

length>200 bp

The weighted mean GenScan P value is a probability for correctly predicting ORFs or partial ORFs and is defined as the (1/Σlᵢ)(Σlᵢ Pᵢ), where “1” is the length of an exon and “P” is the probability or correctness for the exon.

### EXAMPLE 4

This example illustrates the generation of the EST libraries from cDNA prepared from a variety of Glycine max, Oryza sativa, and Zea mays tissue. Seeds are planted in commonly used planting pots and grown in an environmental chamber. Tissue is harvested as follows:
a) For leaf tissue-based cDNA, leaf blades are cut with sharp scissors at seven weeks after planting;
b) For root tissue-based cDNA, roots of seven-week old plants are rinsed intensively with tap water to wash away dirt, and briefly blotted by paper towel to take away free water;
c) For stem tissue-based cDNA, stems are collected seven to eight weeks after planting by cutting the stems from the base and cutting the top of the plant to remove the floral tissue;
d) For flower bud tissue-based cDNA, green and unopened flower buds are harvested about seven weeks after planting;
e) For open flower tissue-based cDNA, completely opened flowers with all parts of floral structure observable, but no siliques are appearing, and are harvested about seven weeks after planting;
f) For immature seed tissue-based cDNA, seeds are harvested at approximately 7-8 weeks of age. The seeds range in maturity from the smallest seeds that could be dissected from siliques to just before starting to turn yellow in color.

All tissue is immediately frozen in liquid nitrogen and stored at −80° C. until total RNA extraction. The stored RNA is purified using Trizol reagent from Life Technologies (Gibco BRL, Life Technologies, Gaithersburg, Md. U.S.A.), essentially as recommended by the manufacturer. Poly A+ RNA (mRNA) is purified using magnetic oligo dT beads essentially as recommended by the manufacturer (Dynabeads, Dynal Corporation, Lake Success, N.Y. U.S.A.).

Construction of plant cDNA libraries is well-known in the art and a number of cloning strategies exist. A number of cDNA library construction kits are commercially available. The Superscript™ Plasmid System for cDNA synthesis and Plasmid Cloning (Gibco BRL, Life Technologies, Gaithersburg, Md. U.S.A.) is used, following the conditions suggested by the manufacturer.

The cDNA libraries are plated on LB agar containing the appropriate antibiotics for selection and incubated at 37° for a sufficient time to allow the growth of individual colonies. Single colonies are individually placed in each well of a 96-well microtiter plates containing LB liquid including the selective antibiotics. The plates are incubated overnight at approximately 37° C. with gentle shaking to promote growth of the cultures. The plasmid DNA is isolated from each clone using Qiaprep plasmid isolation kits, using the conditions recommended by the manufacturer (Qiagen Inc., Santa Clara, Calif. U.S.A.).

The template plasmid DNA clones are used for subsequent sequencing. For sequencing the cDNA libraries, a commercially available sequencing kit, such as the ABI PRISM dRhodamine Terminator Cycle Sequencing Ready Reaction Kit with AmpliTaq® DNA Polymerase, FS, is used under the conditions recommended by the manufacturer (PE Applied Biosystems, Foster City, Calif.). The ESTs of the present invention are generated by sequencing initiated from the 5′ end of each cDNA clone.

A number of sequencing techniques are known in the art, including fluorescence-based sequencing methodologies. These methods have the detection, automation and instrumentation capability necessary for the analysis of large volumes of sequence data. Currently, the 377 DNA Sequencer (Perkin-Elmer Corp., Applied Biosystems Div., Foster City, Calif.) allows the most rapid electrophoresis and data collection. With these types of automated systems, fluorescent dye-labeled sequence reaction products are detected and data entered directly into the computer, producing a chromatogram that is subsequently viewed, stored, and analyzed using the corresponding software programs. These methods are known to those of skill in the art and have been described and reviewed.

The generated ESTs (including any full length cDNA sequences) are combined with ESTs and full length cDNA sequences in public databases such as GenBank. Duplicate sequences are removed; and duplicate sequence identification numbers are replaced. The combined dataset is then clustered and assembled using Pangea Systems tool identified as CAT v.3.2. First, the EST sequences are screened and filtered, e.g. high frequency words are masked to prevent spurious clustering; sequence common to known contaminants such as cloning bacteria are masked; high frequency repeated sequences and simple sequences are masked; unmasked sequences of less than 100 bp are eliminated. The thus-screened and filtered ESTs are combined and subjected to a word-based clustering algorithm which calculates sequence pair distances based on word frequencies and uses a single linkage method to group like sequences into clusters of more than one sequence, as appropriate. Clustered sequence files are assembled individually using an iterative method based on PHRAP/CRAW/MAP providing one or more self-consistent consensus sequences and inconsistent singleton sequences. The assembled clustered sequence files are checked for completeness and parsed to create data representing each consensus contiguous sequence (contig), the initial EST sequences, and the relative position of each EST in a respective contig. The sequence of the 5′ most clone is identified from each contig. The initial sequences that are not included in a contig are separated out. A FASTA file is created consisting of sequences comprising the sequence of each contig and all original sequences which were not included in a contig.

### EXAMPLE 5

cDNA sequences are assembled as above and are translated into all six reading frames. Translations of genes or gene fragments from genomic DNA whose coordinates are determined by Genscan or AAT/NAP are searched against standard or fragment Pfam (version 5.3) profile Hidden Markov Models for transcription factor families as are the cDNA translations. HMMs for transcription factor families in Pfam were rebuilt using HMMER software based on the full alignment provided in Pfam. The E value cutoff is set at 10.

Hidden Markov Models are constructed for transcription factor families not included in the Pfam database by aligning known domains manually. Hidden Markov Models are built using hmmbuild (with and without the -f option) using the HMMER software with the alignments as input. HMM models are calibrated using the HMMER software (hmmcalibrate) with the HMM model as input. Protein data sets are searched with the HMM models using hmmsearch in the HMMER software package version 2.1.1 using default parameters.

Framealign searches are used when known transcription factor domains are not detected by Hidden Markov Models. In these cases, the domains per transcription factor family are listed from the Transfac database. Using Gencore software version 4.5.4 DNA datasets are framealign searched with each domain using an E value cutoff of 1E-3 all other parameters are default. The search results are combined for all domains per family.

Additional transcription factors are found by keyword searches that are carried out against cDNA sequences annotated using the BLAST 2.0 suite of programs with default parameters. Keyword searching is carried out against the top hit (E value better than or equal to 1E-08) using terms indicative of transcription factor families from Table 2.

### DESCRIPTION OF THE TABLES

Table 1 lists the amino acid sequences translated from nucleotide sequences determined to be transcription factors as analyzed in Example 5, above. Column headings are as follows:
SEQ NUM: The entries in the SEQ NUM column refer to the corresponding sequence in the sequence listing.
SEQ ID: The SEQ ID is the name of the sequence.
Family/Method/E value: Entries in this column list the transcription factor family to which the sequence belongs. The families are described in Table 2. The entries also list the method used to determine transcription factor family. “HMM” refers to the Hidden Markov Model method as described in Example 5. “Framesearch” refers to the framealign search method described in Example 5 and “keyword” refers to BLAST annotation followed by keyword searching as described in Example 5. The E value for each of the methods is also listed in this column. E value is defined as the expectation E (range 0 to infinity) calculated for an alignment between the query sequence and a database sequence can be extrapolated to an expectation over the entire database search, by converting the pairwise expectation to a probability (range 0-1) and multiplying the result by the ratio of the entire database size (expressed in residues) to the length of the matching database sequence. In detail:
   E_database=(1−exp(−E)) D/d where D is the size of the database; d is the length of the matching database sequence; and the quantity (1−exp(−E)) is the probability, P, corresponding to the expectation E for the pairwise sequence comparison.

Table 2 lists transcription factor families, a brief description of each, and other related families. Column headings are as follows:
Transcription Factor Family: Entries in this column list the transcription factor families as listed in the Pfam database, Transfac, or PROSITE.

Family Name and Domain Description: Entries in this column describe the transcription factor families listed in column 1. These descriptions are from the Pfam database, Transfac, or PROSITE.

TABLE 2

| Trascription | |
|---|---|
| Factor Family | Family Name and Domain Description |
| AP2 | This 60 amino acid residue domain can bind to DNA -- |
| | this domain is plant specific -- members of this family |
| | are suggested to be related to pyridoxal phosphate- |
| | binding domains such as found in aminotran 2 - |
| | ethylene response (inducible). Examples: ethylene- |
| | responsive element binding proteins (EREBPs) & E. |
| | coli universal stress protein UspA |
| ANK | Ankyrin repeat. Some Ankyrin-only proteins will |
| | interact with rel-ankyrin proteins to inhibit DNA |
| | binding activity. Examples: IkB α, γ, β and cactus. |
| ARF | Auxin response factor -- plant specific. Not in Pfam- |
| | not to be confused with similarly named ADP- |
| | ribosylation factor (GTP binding protein) that is |
| | listed as ARF in Pfam. |
| ARID | AT-Rich Interaction Domain - DNA-binding. Exam- |
| | ples: Structural homology with T4 RNase H, E. coli |
| | endonuclease III & Bacillus subtilis DNA polymerase I |
| AT-hook | The AT-hook is an AT-rich DNA-binding motif |
| | that was first described in mammalian high- |
| | mobility-group non-histone chromosomal protein |
| | HMG-I/Y. It is necessary and sufficient for binding |
| | to the narrow minor groove of stretches of AT-rich |
| | DNA via a conserved nine amino acid peptide |
| | (KRPRGRPKK). Many of the AT-hook DNA-binding |
| | motif proteins have been shown to have an effect |
| | on the structure and architecture of chromatin at |
| | levels beyond the action of the basis histones. |
| | The have been shown to also play a role in |
| | transcription regulation by acting as cofactors. |
| 14-3-3 | The 14-3-3 proteins are a family of closely related |
| | acidic homodimeric proteins of about 30 Kd. The |
| | GF14 (G-Box Factor 14-3-3 Homolog) family is a |
| | group of proteins similar to 14-3-3 proteins that |
| | bind G-box oligonucleotides in promoters to regulate |
| | transcription. |
| B3 | Similar to ARF - plant specific. Not in Pfam. Binds |
| | DNA directly. |
| BAH | Bromo-adjacent homology. Appears to act as a pro- |
| | tein-protein interaction module specialized in gene |
| | silencing. It might play an important role by linking |
| | DNA methylation, replication and transcriptional |
| | regulation. Examples: DNA (cytosine-5) methyl- |
| | transferases & Origin recognition complex 1 (Ore1) |
| | proteins. |
| basic | This basic domain is found in the MyoD family |
| | of muscle specific proteins that control muscle |
| | development. The bHLH region of the MyoD family |
| | includes the basic domain and the Helix-loop-helix |
| | (HLH) motif. The bHLH region mediates specific |
| | DNA binding with 12 residues of the basic domain |
| | involved in DNA binding. The basic domain forms |
| | an extended alpha helix in the structure. |
| BPF-1 | The parsley BPF-1 protein (Box P-binding factor) |
| | was identified as a transcription factor that bound |
| | the promoter of phenylalanine ammonia lyase |
| | (PAL1) in response to a fungal elicitor. An |
| | Arabidopsis binding HPPBF-1 (H-protein promotoer |
| | binding factor-1), was found to regulate light- |
| | dependent expression of the H subunit of glycine |
| | decarboxylase, a mitochondrial enzyme complex |
| | involved in photorespiration. |
| bromodomain | About 70 amino acids -- Exact function |
| | of this domain is not yet known but it is thought |
| | to be involved in protein-protein interactions |
| | and it may be important for the assembly or activity |
| | of multicomponent complexes involved in transcrip- |
| | tional activation. Examples: Mammalian CREB-bind- |
| | ing protein; also found in many chromatin associated |
| | proteins -- bromodomains can interact specifically |
| | with acetylated lysine. |
| BTB | Named for BR-C, ttk and bab -- approximately 115 |
| | amino acids. The POZ or BTB domain is also known |
| | as BR-C/Ttk or ZiN Found primarily in zinc finger |
| | proteins -- present near the N-terminus of a fraction |
| | of zinc finger (zf-C2H2) proteins. The BTB/POZ |
| | domain mediates homomeric dimerization and in some |
| | instances heteromeric dimerization -- inhibits |
| | the interaction of their associated finger regions |
| | with DNA -- shown to mediate transcriptional |
| | repression and to interact with components of histone |
| | deacetylase co-repressor complexes. Other |
| | Examples: Drosophila bric a brac protein plus an |
| | estimated 40 members in Drosophila. |
| BZIP | Basic region mediating sequence-specific DNA- |
| | binding followed by a leucine zipper required |
| | for dimerization -- family is quite large. Examples: |
| | Fos, Jun, CRE, & Arabidopsis G-box binding factors. |
| | GBF. |
| CBFD, NFYB, | Histone-like transcription factors (CBF/NF-Y) |
| HMF | and archeal histones CCAAT-binding factor |
| | (CBF). Heteromeric transcription factor that consists |
| | of two different components, both needed |
| | for DNA-binding. First subunit of CBFD (NF-YB) |
| | binds DNA (protein of 116 to 210 amino-acid |
| | residues); the second subunit of CBFD (NF-YA) |
| | contains an N-terminal subunit-association |
| | domain and a C-terminal DNA recognition domain |
| | (a protein of 265 to 350 amino-acid residues). |
| | Other Examples: histone-like subunits of |
| | transcription factor IID. |
| chromo | CHRromatin Organization MOdifier -- about 60 |
| | amino acids Originally found in proteins that |
| | modify the structure of chromatin to the condensed |
| | morphology of heterochromatin (Drosophila |
| | modifiers or variegation). Examples: Fission yeast |
| | swi6 (repression of the silent mating-type |
| | loci mat2 and mat3), Drosophila protein Su(var)3-9 |
| | (a suppressor of position-effect variegation), |
| | & mammalian DNA-binding/helicase proteins |
| | CHD-1 to CHD-4. |
| chromo shadow | This domain is distantly related to chromo. This |
| | domain is always found in association with a |
| | chromo domain although not all chromo domain |
| | proteins contain the chromo shadow. Examples: |
| | Fission yeast swi6 (repression of the silent |
| | mating-type loci mat2 and mat3). |
| Copper-first | Some fungal transcription factors contain |
| | a N-terminal domain that seems to be involved in |
| | copper-dependent DNA-binding -- undergo a |
| | conformational change in presence of copper. |
| | Examples: Yeast ACE1 (or CUP2) and Candida |
| | glabrata AMT1 that regulate the expression of |
| | the metallothionein genes -- Yarrowia lipolytica |
| | copper resistance protein CRF1. |
| CSD | Cold shock domain -- about 70 amino acids. Binds |
| | to the CCAAT-containing Y box and the B |
| | box. Binds to cold tolerance gene promotoers in |
| | bacteria. Examples: E. coli protein CS7.4 |
| | (gene cspA) that is induced in response to low |
| | temperature & Bacillus subtilis cold-shock |
| | proteins cspB and cspC. |
| Ctf/nf1 | Nuclear factor 1 (MF-1) or CCAAT box-binding |
| | transcription factor (CTF) (also known as |
| | TGGCA-binding proteins) are a family of vertebrate |
| | nuclear proteins which recognize and bind, as |
| | dimers, the palindromic DNA sequence 5′- |
| | TGGCANNNTGCCA-3′. CTF/NF-1 |
| | binding sites are present in viral and cellular |
| | promoters and in the origin of DNA replication |
| | of Adenovirus type 2. |
| Dm-domain | The DM domain is named after dsx and mab-3 -- |
| | dsx contains a single amino-terminal DM domain, |
| | whereas mab-3 contains two amino-terminal |
| | domains. The DM domain has a pattern of |
| | conserved zinc chelating residues C2H2C4. |
| | The dsx DM domain has been shown to dimerize |
| | and bind palindromic DNA. |
| Dof | Dof proteins are a family of TFs that share a unique |
| | DNA-binding domain of ˜52 aa. May form |
| | a single zinc-finger that is essential for DNA |
| | recognition. Plant specific and have various |
| | roles in the cell. Found in both monocots |
| | and dicots. |
| DPB | Described by Mendel as the DNA-binding protein |
| | (DBP) family, a collection of miscellaneous proteins |
| | that have been functionally identified by their |
| | ability to physically bind to DNA via a DNA- |
| | binding domain. Here, includes the remorin like DNA- |
| | binding proteins. Also see TEO which describes |
| | the PCF1/2 like TFs. |
| ENBP | ENBP1 (early nodulin gene-binding protein 1), binds |
| | to an AT-rich regulatory element of psENOD12b |
| | to regulate its expression upon infection of plant |
| | root hairs by nitrogen-fixing bacteria. ENBP1 |
| | and ENBP1-like transcription factors are probably |
| | involved in general cellular processes, others |
| | than in a symbiotic context. |
| Ets | Ets transcription factors are nuclear effectors |
| | of the Ras-MAP-kinase signaling pathway. |
| | Avian leukemia virus E26 is a replication defective |
| | retrovirus that induces a mixed erythroid/ |
| | myeloid leukemia in chickens. E26 virus carries |
| | two distinct oncogenes, v-myb and v-ets. |
| | The ets portion of this oncogene is required |
| | for the induction of erythroblastosis. V-ets and |
| | c-ets-1, its cellular progenitor, have been |
| | shown to be nuclear DNA-binding proteins. |
| Fork_head | About 100 amino-acid residues, also known |
| | as the “winged helix” - present in some |
| | eukaryotic transcription factors - involved in |
| | DNA-binding. Examples: Drosophila forkhead (fkh), |
| | mammalian transcriptional activators HNF-3-alpha, |
| | -beta, and -gamma, human HTLF, Xenopus XFKH1, |
| | yeast HCM1, yeast FKH1. |
| GATA | GATA family of transcription factors are proteins |
| | that bind to DNA sites with the consensus sequence |
| | (A/T)GATA(A/G). Contain a pair of highly |
| | similar ‘zinc finger’ type domains. Examples: |
| | GATA 1-4 are TF found in mammals; they regulate |
| | development in certain cell types by binding to the |
| | GATA promoter region of globulin genes, & others. |
| | Note: similar single ‘zinc finger’ domain |
| | protein is involved in positive and negative nitrogen |
| | metabolism gene regulation in fungus and yeast and |
| | also Neurospora crassa light regulated genes. |
| Gld | A domain with limited amino acid similarity to the |
| | TEA DNA binding domain found in a number of |
| | regulatory genes from fungi, insects, and |
| | mammals. This domain is predicted to form two |
| | alpha helices with sequence similarity to two |
| | alpha helices of the TEA domain that are |
| | implicated in DNA binding. These proteins are |
| | not picked up by Pfam's TEA model. Found in |
| | some response_reg proteins. Examples: |
| | ARR, AT1; both in Arabidopsis. Golden2 in maize. |
| HhH | Helix-hairpin-helix motif - multiple domains found |
| | in a protein. These HhH motifs bind DNA in a non- |
| | sequence-specific manner. Examples: Rat pol beta, |
| | endonuclease III, AlkaA, & the 5′ nuclease |
| | domain of Taq pol 1. |
| Hist_deacetyl | Regulation of transcription is caused in part |
| | by reversibly acetylating histones on several lysine |
| | residues. Histone deacetylases catalyze the removal |
| | of the acetyl group. |
| HLH | Helix-loop-helix domain - 40 to 50 amino acid |
| | residues. Two amphipathic delices joined by a variable |
| | length linker region that could form a loop. This |
| | ‘helix-loop-helix’ (HLH) domain mediates protein |
| | dimerization -- most of these proteins have an extra |
| | basic region of about 15 amino acid residues |
| | adjacent to the HLH domain which specifically |
| | binds to DNA - members of the family are referred |
| | to as basic helix-loop-helix proteins (bHLH) -- |
| | bind E boxes -- dimerization is necessary but |
| | independent of DNA binding -- proteins without |
| | basic region act as repressors since they are |
| | unable to bind DNA but do dimerize. Examples: |
| | Myc (oncogene), Myo (muscle differentiation), |
| | Maize anthocyanin regulatory proteins, and other |
| | cellular differentiation TFs. |
| HMG_box | High mobility group; relatively low molecular weight |
| | non-histone components in chromatin Known to |
| | bind to nucleosomes in active chromatin - |
| | thought to be involved in chromatin formation. |
| HMG14_17 | High mobility group. HMG14 and HMG17 are two |
| | related proteins of about 100 amino acid |
| | residues that bind to the inner side of the |
| | nucleosomal DNA thus altering the interaction |
| | between the DNA and the histone octamer. These two |
| | proteins may be involved in the process that |
| | maintains transcribable genes in a unique |
| | chromatin conformation. |
| Homeobox | Master control homeotic genes that determine body |
| | plan -- 60-residue motif - subfamilies named |
| | for 3 Drosophila gene families. Play an important |
| | role in development - most are known to be |
| | sequence-specific DNA-binding transcription factors. |
| | The domain binds DNA through a helix-turn-helix |
| | (HTH) structure. -- Homeobox is a 3-element |
| | fingerprint that provides a signature for the |
| | homeobox domain of homeotic proteins. Examples: |
| | Drosophila hox proteins: antennapedia (Antp), |
| | abdominal-A (abd-A), deformed (Dfd), proboscipedia |
| | (pb), sex combs reduced (scr), and ultrabithorax |
| | (ubx) which are collectively known as the |
| | ‘antennapedia’ subfamily; the engrailed |
| | subfamily defined by engrailed (en) which specifies |
| | the body segmentation pattern and is required for |
| | the development of the CNS; and the paired gene |
| | subfamily. |
| Histone | Histone protein is uniqie to eukaryotes -- an |
| | octamer is assembled to form chromatin with 146 |
| | base pairs of DNA organized into a superhelix |
| | around a histone octomer to create a nucleosome |
| | (‘beads on a string’). Examples: H2A, H2B, H3, |
| | & H4. |
| HSF_DNA- | Heat shock factor (HSF) is a DNA-binding protein |
| binding | that specifically binds heat shock promoter elements |
| | (HSE). HSF is expressed at normal temperatures but |
| | is activated by heat shoch or chemical stresses. |
| IAA | The Aux-IAA proteins were indentified as a class |
| | of short-lived, nuclear localized proteins that |
| | are rapidly transcriptionally induced in response to |
| | auxin. These proteins contain four highly cconserved |
| | domains (boxes I, II, III, IV)- this model |
| | covers boxes III and IV. See ARF family in |
| | this document for related proteins. |
| IBR | The IBR (In Between Ring fingers) domain is found |
| | to occur between pairs of ring fingers (Zf-C3HC4). |
| | The function of this domain is unknown. |
| irf | This family of transcription factors is improtant in |
| | the regulation of interferons in response to infection |
| | by virus and in the regulation of interferon-inducible |
| | genes. Three of the five conserved tryptophan residues |
| | bind to DNA. |
| K-box | K-box region is commonly found in associated with |
| | SRF-type transcription factors. The K-box is a possible |
| | coiled-coil structure. Possible role in multimer |
| | formation. Examples: PISTILLATA (PI) gene of |
| | Arabidopsis causes homeotic conversion of petals to |
| | sepals and of stamens to carpels & SRF (Serum |
| | response factor) binds the serum response element. |
| KRAB | The KRAB domain (or Kruppel-associated box) is |
| | present in about a third of zinc finger proteins |
| | containing C2H2 fingers. The KRAB domain is found |
| | to be involved in protein-protein interactions. |
| LIM | Cysteine-rich domain of about 60 amino-acid residues. |
| | Generally occurs as two tandem copies in proteins - |
| | in the LIM domain, there are seven conserved cysteine |
| | residues and a histidine -- the LIM domain binds two |
| | zinc ions -- LIM does bot bind DNA, rather it seems |
| | to act as interface for protein-protein interaction. |
| | Examples: Pollen specific protein (SF3), Mammalian |
| | zinc absorption protein, Vertebrate paxillin |
| | (cytoskeletal focal adhesion protein), Plaque |
| | adhesion protein, and several homeotic proteins. |
| Linker_histone | Member of histone octamer - see histone. |
| | Examples: H1, H5 |
| MADS | See SRF-TF |
| Myb_DNA- | This family contains the DNA-binding domaines |
| binding | from the Myb proteins, as well as the SANT domain |
| | family. Retroviral oncogene v-myb, and its cellular |
| | counterpart c-myb, encode nuclear DNA-binding |
| | proteins that specifically recognize the sequence |
| | YAAC(G/T)G. Examples: Maize C1 protein |
| | (anthocyanin biosynthesis). Maize P protein |
| | (regulates the biosynthetic pathway of |
| | a flavonoid-derived pigment in certain floral tissues), |
| | Arabisopsis GL1 (required for the initiation of |
| | differentiation of leaf hair cells/trichomes), Yeast |
| | txn & telomere length proteins. |
| Myc N Term | Myc amino-terminal region. The myc family belongs |
| | to the basic helix-loop-helix leucine zipper class of |
| | transcription factors. Myc forms a heterodimer with |
| | Max, and this complex regulates cell growth through |
| | direct activation of genes involved in cell replication. |
| | c-Myc can also repress the transcription of specific |
| | genes. |
| NAM | The NAM (no apical meristem) family is a group |
| | of transcription factors that share a highly conserved |
| | N-terminal domain of about 150 amino acids, |
| | designated the NAC domain (NAC stands for Petunia, |
| | NAM, and Arabisopsis, ATAF1, ATAF2 and CUC2). |
| | Present in monocots and dicots. Probably have |
| | roles in the regulation of embryo and flower |
| | development. Plant specific. |
| NAP_FAMILY | Nucleosome assembly protein (NAP) -- histone |
| | chaperonel May be involved in regulating gene |
| | expression as a result of histone accessibility. NAP-2 |
| | (human NAP clone) can interact with both core and |
| | linker histones and recombinant NAP-2 can transfer |
| | histones onto naked DNA templates. |
| P53 | The p53 tumor antigen is a protein found in increased |
| | amounts in a wide variety of transformed cells. p53 is |
| | probably involved in cell cycle regulation, and may |
| | be trans-activator that acts to negatively regulate |
| | cellular division by controlling a set of genes required |
| | for this process. |
| Pax | “paired box” domain -- a 124 amino-acid conserved |
| | domain -- generally located in the N-terminal section |
| | of the proteins -- function of this conserved domain |
| | is not yet known. In some of the pax proteins, there |
| | is a homeobox domain upstream of the paired box. |
| | Examples: Drosophila segmentation pair-rule class |
| | protein paired (prd), Drosophila proteins Pox-meso |
| | and Pox-neuro, the PAX proteins. |
| PHD | Zinc finger-like motif. Regulate the expression of the |
| | homeotic genes through a mechanism thought to |
| | involve some aspect of chromatin structure. |
| | Speculate that the PHD-fingers are protein-protein |
| | interaction domains or that they recognize a |
| | family of related targets in the nucleus such |
| | as the nucleosomal histone tails. |
| POU | ‘POU’ (pronounced ‘pow’) domain -- a 70 to 75 |
| | amino-acid region found upstream of a homeobox |
| | domain in some eukaryotic transcription factors. |
| | It is thought to confer high-affinity site- |
| | specific DNA-binding and to mediate cooperative |
| | protein-protein interaction on DNA. Examples: |
| | Oct genes (bind to immunoglobulim promoter |
| | octomer region to activate genes), Neuronal |
| | development genes, & C. elegans development genes |
| Protamine_p2 | Protamine P2 can substitute for histones |
| | in the chromatin of sperm. |
| Response_reg | This domain receives the signal from the sensor |
| | partner in bacterial two-component systems. It |
| | is usually found N-terminal to a DNA binding |
| | effector domain (e.g. GLD). |
| Rhd | Conserved domain in a family of eukaryotic |
| | transcription factors with basic impact on |
| | oncogenesis, embryonic development and |
| | differentiation including immune response |
| | and acute phase reaction -- composed of |
| | two structural domains, the N-terminal region is |
| | similar to that found in P53, whereas the C terminal |
| | region is an immunoglobulin-like fold. Examples: |
| | NF-kappa-B, RelB, Drosophila Dif. |
| Runt | New family off heteromeric TFs. |
| Scan | The SCAN domain (named after SRE-ZBP, CTfin51, |
| | AW-1 and Number 18 cDNA) is found in several |
| | zf-c2h2 proteins. This conserved domain has |
| | been shown to be able to mediate homo- and |
| | hetero-oligomerisation. |
| SCR | The Arabidopsis SCARECROW gene regulates an |
| | assymetric cell division essential for proper radial |
| | organization of root cell layers. It was tentaively |
| | described as a transcription factor based on the |
| | presence of homopolymeric stretches of several |
| | amino acids, the presence of a basic domain |
| | similar to that of the basic-leucine zipper family |
| | of transcription factors, and the presence of |
| | leucine heptad repeats. Two SCARECROW |
| | homologs, RGA and GA1, are involved in the |
| | gibberellin signal transduction pathway. |
| SBPB | A new family of DNA binding proteins (putative |
| | transcriptional regulators) called squamosa |
| | promoter binding proteins of SBPs that |
| | potentially regulate floral transition. |
| | The SBPs possess a bipartite nuclear localization |
| | signal, a putative acidic activation domain |
| | and a so-called SBP-box DNA binding domain motif |
| | that does not show similarity to any known DNA |
| | binding motif. |
| SET | SET (Suvar-3-9, Enhancer-of-zeste, & Trithorax) |
| | domains appear to be protein-protein interaction |
| | domains. It has been demonstrated that SET |
| | domains mediate interactions with a family of |
| | proteins that display similarity with dual-specificty |
| | phosphatases (dsPTPases). Link SET-domain |
| | containing ccomponents of the epigenetic |
| | regulatory machinery with signalling pathways |
| | involved in growth and differentiation. Examples: |
| | ASH1 protein contains a SET domain and a PHD |
| | finger (required for stable patterns of |
| | homeotic gene expression in Drosophila). |
| SNF2_N | SNF2 and “others” N-terminal domain. Examples: |
| | This domain is found in proteins involved in |
| | a variety of processes including transcription |
| | regulation (e.g., SNF2, STH1, brahma, MOT1), |
| | DNA repair (e.g., ERCC6, RAD16, RAD5), |
| | DNA recombination (e.g., RAD54), & chromatin |
| | unwinding (e.g., ISW1) as well as a variety |
| | of other proteins with little functional |
| | information (e.g., lodestar, ETL1). |
| SRF-TF | 56 amino-acid residues - function as dimers -- |
| (MADS) | commonly homeotic proteins. Examples: Human |
| | serum response factor (SRF), a ubiquitous |
| | nuclear protein important for cell proliferation |
| | and differentiation; homeotic proteins involved in |
| | control of floral development; yeast arginine |
| | metabolism regulation protein I, & yeast mating |
| | type specific genes. |
| Stat | STAT proteins (Signal Transducers and Activators of |
| | Transcription) are a family of transcription |
| | factors that are specifically activated to regulate |
| | gene transcription when cells encounter cytokines |
| | and growth factors. STAT proteins also include |
| | an SH2 domain. |
| TBP | Transcription factor TFHD (or TATA-binding protein, |
| | TBP). General factor that plays a major role in |
| | the activation of eukaryotic genes transcribed by RNA |
| | polymerase II - binds the TATA box -- C-terminal |
| | domain of about 180 residues contains two conserved |
| | repeats of 1 77 amino-acid region. Generates |
| | a saddle-shaped structure that sits astride |
| | the DNA. |
| t-box | About 170 to 190 amino acids, known as the T-box |
| | domain. First found in mouse T locus (Brachyury) |
| | protein, a transcription factor involved in |
| | mesoderm differentiation. Essential in tissue |
| | specification, morphogenesis and organogenesis |
| Tea | A DNA-binding region of about 66 to 68 amino acids |
| | that has been found in the N-terminal section |
| | of several regulatory proteins. Examples: |
| | Mammalian enhancer factor TEF-1, Drosophila |
| | scalloped protein (gene sd), Emericella nidulans |
| | regulatory protein abaA, yeast trans-acting |
| | factor TEC1, C. elegans hypothetical protein |
| | F28B12.2. |
| TEO | The founding members of this gene family are |
| | tesostine-branched1 of maize and cycloidea |
| | of Antirrhinum (snapdragon), both of which |
| | are involved in the control of plant form |
| | and structure. They have limited similarity |
| | to the rice DNA binding proteins PCF1 and |
| | PCF2. All share a predicted basic-helix-loop-helix |
| | domain, TCP, which has been shown to be required for |
| | DNA binding of PCF1 and PCF2. |
| TFIIS | Transcription factor S-II (TFIIS). Necessary for |
| | efficient RNA polymerase II transcription elongation, |
| | past template-encoded pause sites. TFIIS shows |
| | DNA-binding activity only in the presence of |
| | RNA polymerase II. Contains four cysteines that |
| | bind a zinc ion and fold in a conformation termed |
| | a ‘zinc ribbon’. Examples: also includes |
| | the eukaryotic and archebacterial RNA polymerase |
| | subunits of the 15 Kd/M family, African |
| | swine fever virus protein I243L, & Vaccinia |
| | virus RNA polymerase. |
| Trihelix | Plant specific domain involved in light response -- |
| | plant specific; not in Pfam. |
| Transcript_fac2 | Transcription factor TFIIB repeat. |
| WRKY | ˜50-60 aa domain. Often repeated within a WRKY |
| | protein, butt it may also be present as a single |
| | copy. WRKY proteins contain several general features |
| | typical of transcription factors, like putative |
| | nuclear localization signals and transcription |
| | activation domains. Founding memebers are ABF1 |
| | and ABF2 proteins. May be involved in regulation |
| | of sporamin and alpha-amy genes. May also play |
| | a role in the signal transduction pathway |
| | that leads to pathogenesis-related (PR) |
| | gene activation in response to pathogens. |
| ZF-B box | B-box zinc finger. |
| ZF-C2H2 | The first zinc finger class to be characterized -- |
| | the first pair of zinc coordinating residues |
| | are cysteines, while the second pair are histidines. |
| | A number of experimental reports have demonstrated |
| | the zinc- dependent DNA or RNA binding property |
| | of some members of this class. Examples: Mammalian |
| | transcription factors Sp1-4, Xenopus transcription |
| | factor TFIIA, & Drosophila Hunchback and Kruppel |
| Zf-C3HC4 | Conserved cysteine-rich domain of 40 to 60 residues |
| | (called C3HC4 zinc-finger or ‘RING’ finger) that |
| | binds two atoms of zinc, and is probably involved |
| | in mediating protein-protein interactions. |
| ZF-C4 | Conserved cysteine-rich DNA-binding region of some |
| | 65 residues. Almost always the DNA-binding domain |
| | of a nuclear hormone receptor. Receptors for steroid, |
| | thyroid, and retinoid hormones belong to a family |
| | of nuclear trans-acting transcriptional regulatory |
| | factors. These proteins regulate diverse biological |
| | processes such as pattern formation, cellular |
| | differentiation and hormeostasis. |
| ZF-CCCH | Zinc finger |
| ZF-CCHC | A family of CCHC zinc fingers, mostly from retroviral |
| | gag proteins (nucleocapsid). Prototype structure |
| | is from HIV. Also contains members involved in |
| | eukaryotic gene regulation, such as C. elegans GLH-1. |
| | Structure is an 18-residue zinc finger. |
| ZF-CHC2 | CHC2 zinc finger |
| ZF-CONSTANTS | CONSTANTS family zinc finger. So far only reported |
| | in plants. CONSTANTS (CO) gene of Arabidopsis |
| | promotes flowering. Some transgenic plants containing |
| | extra copies of CO flowered earlier than wild type, |
| | suggesting CO activity is limiting on flowering time. |
| | Double mutants were constructed containing CO and |
| | mutations affecting gibberellic acid responses, |
| | meristem identity, or phytochrome function, and their |
| | phenotypes suggested a model for the role of CO in |
| | promoting flowering. |
| Zf-C2HC | A DNA-binding zinc finger domain. Examples: human |
| | myelin transcription factor (Myt). C. elegans |
| | hypothetical protein F52F12.6, |
| ZF-MYND | DNA-binding domain found in Drosophila DEAF-1 |
| | protein that binds to a 120 bp homeotic response |
| | element. |
| ZN_CLUS | A cysteine-rich region that binds DNA in a zinc- |
| | dependent fashion. Found in fungal transcriptional |
| | activator proteins. It has been shown that this region |
| | forms a binuclear zinc cluster where six conserved |
| | cysteines bind two zinc cations. |
| ZZ | New putative zinc finger in dystrophin and other |
| | proteins. Binds calmodulin. DNA-binding not yet |
| | shown. |
| ZF-NF-X1 | Cysteine-rich sequence-specific DNA-binding protein. |
| | Interacts with the conserved X-box motif of the |
| | human major histocompatability complex class II |
| | genes via a repeated Cys-His domain and functions |
| | as a transcriptional repressor. |

All publications and patent applications cited herein are incorporated by reference in their entirely to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A substantially purified nucleic acid molecule comprising a nucleic acid sequence selected from the group consisting of SEQ ID NO: 1-5429, SEQ ID NO: 10859-15800, SEQ ID NO: 20743-23549, and SEQ ID NO: 26357-29936 or complements thereof.

2. A substantially purified protein or fragment thereof comprising an amino acid sequence selected from the group consisting of SEQ ID NO: 5430-10858, SEQ ID NO: 15801-20742, SEQ ID NO: 23550-26356, and SEQ ID NO: 29937-33516 or fragment thereof.

3. A method of producing a plant containing an overexpressed plant transcription factor comprising:
(a) transforming said plant with a functional first nucleic acid molecule, wherein said first nucleic acid molecule comprises a promoter region, wherein said promoter region is linked to a structural region, wherein said structural region comprises a second nucleic acid molecule having a nucleic acid sequence selected from the group consisting of SEQ ID NO: 1-5429, SEQ ID NO: 10859-15800, SEQ ID NO: 20743-23549, and SEQ ID NO: 26357-29936; wherein said structural region is linked to a 3′ non-translated sequence that functions in the plant to cause termination of transcription of transcription and addition of polyadenylated ribonucleotides to a 3′ end of a mRNA molecule; and wherein said function first nucleic acid molecule results in overexpression of the plant transcription factor; and
(b) growing said plant.

4. A method for determining a level or pattern of a plant transcription factor in a plant cell or plant tissue comprising:
(a) incubating, under conditions permitting nucleic acid hybridization, a marker nucleic acid molecule, the marker nucleic acid molecule selected from the group of marker nucleic acid molecules which specifically hybridize to a nucleic acid molecule having the nucleic acid sequence selected from the group consisting of SEQ ID NO: 1-5429, SEQ ID NO: 10859-15800, SEQ ID NO: 20743-23549, and SEQ ID NO: 26357-29936 or complements thereof or fragments of either, with a complementary nucleic acid molecule obtained from the plant cell or plant tissue, wherein nucleic acid hybridization between the marker nucleic acid molecule and the complementary nucleic acid molecule obtained from the plant cell or plant tissue permits the detection of an mRNA for the enzyme;
(b) permitting hybridization between the marker nucleic acid molecule and the complementary nucleic acid molecule obtained from the plant cell or plant tissue; and
(c) detecting the level or pattern of the complementary nucleic acid, wherein the detection of the complementary nucleic acid is predictive of the level or pattern of the plant transcription factor.
